(19) United States
(12) Patent Application Publication
(10) Pub. No.: US 2011/0046204 A1
(43) Pub. Date: Feb. 24, 2011

(54) Human Zip1, Zinc and Citrate for Prostate Cancer Screening

(75) Inventors: Leslie C. Costello, Severna Park, MD (US); Renty B. Franklin, Burtonsville, MD (US)

Correspondence Address: Frederick W. Gibb, III, Esq.;Gibb I.P. Law Firm, LLC, 844 West Street, Suite 100, ANNAPOLIS, MD, 21401, US

(73) Assignee: University of Maryland, Balitmore

(21) Appl. No.: 12/223,152

(22) PCT Filed: Jan. 23, 2007
(86) PCT No.: PCT/US07/01681

§ 371 Date: May 18, 2010

Related U.S. Application Data
(60) Provisional application No. 60/761,258, filed on Jan. 23, 2006.

(51) Int. Cl. A61K 48/00; C12Q 1/68; G01N 33/566; G01N 33/50; C12Q 1/02; C12Q 1/527; C07K 16/18; C12N 15/63; C12N 5/071; A61P 35/00; G01N 23/223

(52) U.S. Cl. 514/44 R; 435/6; 435/7.21; 436/501; 436/81; 435/29; 436/79; 436/80; 436/129; 435/4; 530/387.9; 530/389.1; 435/320.1; 435/375; 378/45

(57) ABSTRACT

The present invention provides methods of detecting prostate cancer employing biomarkers, including hZIP1, zinc and citrate. Also provided are antibodies to detect hZIP1 protein or peptides and an expression vector comprising a genetic sequence effective to increase uptake of zinc into a prostate cell upon expression thereof. Furthermore, methods of treating prostate cancer and of increasing uptake of zinc into a prostate cell are provided.

### FEDERAL FUNDING LEGEND

This invention was produced using funds obtained through a National Cancer Institute CA079903. Consequently, the Federal government has certain rights in this invention.

### BACKGROUND OF THE INVENTION

1. Field of the Invention

The present invention is related to the fields of medicine and oncology. More specifically the invention relates to modulation of the human ZIP1 gene. The invention further relates to determination of zinc and citrate levels in the detection of prostate cancer.

2. Description of the Related Art

It is critical to identify individuals at risk for clinically manifested prostate cancer and to detect early prostate malignancy to prevent the development of advanced stage prostate cancer. Recent advances have made the treatment of early stage prostate cancer very effective. Therefore a focus on the methods for early detection is most desirable in combating prostate cancer.

The combination of prostate specific antigen (PSA) testing and digital rectal examination is currently the primary step for prostate cancer screening/detection. PSA testing requires invasive blood sampling followed by assaying generally by a commercial laboratory. However, a number of mitigating factors impact the reliability of PSA detection for prostate cancer. Moreover, about 60% of the cases of men referred to biopsy due to predominantly elevated PSA turn out to be negative by histopathological examination, which reflects high false-positive results that plague the use of PSA. Currently, the pathologist's result is the “gold standard” for prostate cancer diagnosis.

The normal peripheral zone glandular epithelial cells of the human prostate gland possess a unique highly-specialized function and capability of accumulating enormously high levels of zinc and citrate. This results in two important effects: a metabolic effect and a proliferative effect. Its metabolic effect is the inhibition of citrate oxidation that is essential for the prostate function of production and secretion of high levels of citrate and its inhibition of terminal oxidation. This has a bioenergetic cost in that the inhibition of citrate oxidation results in a ˜60% loss of ATP production that would arise from complete glucose oxidation. Consequently, zinc-accumulating citrate-producing cells are energy-inefficient cells. A second effect of zinc is its inhibition of prostate cell proliferation.

In contrast to normal epithelial cells, malignant epithelial cells undergo a metabolic transformation that includes losing the ability to accumulate zinc and citrate. Malignant prostate cells must replace the metabolic pathways associated with net citrate production with metabolic relationships that are suitable for their malignant existence. That the malignant prostate cells in situ never exist as zinc-accumulating, citrate-producing cells is evidence of the incompatibility of high zinc accumulation and net citrate production for their existence. Their metabolic transformation to energy-efficient citrate-oxidizing cells that have lost the ability to accumulate zinc provides their metabolic/bioenergetic requirements of malignancy. Also, the apoptotic influence of zinc is eliminated, which permits the proliferation of the malignant cells.

Established clinical and experimental studies provide evidence that decreased zinc and citrate levels are characteristics that distinguish malignant prostate (peripheral zone) from normal peripheral zone. Moreover, even transition zone malignancy exhibits the depletion of citrate levels (1). The clinical and research community has largely ignored the significance and implications of the zinc-citrate relationships in the pathogenesis of prostate cancer.

An endorectal coil and 1H magnetic resonance spectroscopic imaging (MRSI) to determine citrate levels for in situ identification of malignant loci in the peripheral zone was developed in 1989. Two important points that became evident from magnetic resonance spectroscopic imaging reports are that the citrate levels of malignant loci are significantly lower than corresponding normal peripheral zone; and more importantly that the malignant tissue never exhibits a high citrate level (2). Moreover, this citrate relationship also exists in malignancy associated with the transition zone. In addition, the decrease in citrate occurs early in malignancy, which is verified by the recent MRS studies (3-5).

As in the case of citrate, a consistent decrease in zinc is demonstrated in different reports by investigators employing different populations and tissue samples and involving various stages of malignancy (5-6). The individual zinc levels of malignant prostate tissue from different subjects were found to be low as compared to the zinc levels in normal prostate or benign hyperplastic prostate tissue samples. It also was shown that the zinc levels in expressed prostatic fluid from cancer subjects are always low and reflect the same relationship as the changes in the prostate tissue. Moreover, in recent in situ studies it was shown that normal peripheral zone glandular epithelium exhibits high cellular zinc levels and that the adenocarcinomatous glands exhibit a depletion of zinc. The decrease in zinc was apparent in highly-differentiated and in de-differentiated adenocarcinomatous glands.

Human ZIP1 (hZIP1) is an important transporter for the uptake and accumulation of zinc in prostate cells (3, 7-8). Transfected PC-3 cells that overexpress hZIP1 exhibit increased zinc uptake. Downregulation of hZIP1 by treating PC-3 cells with hZIP1 antisense oligonucleotide resulted in decreased zinc uptake. This finding is especially relevant, as a downregulation of hZIP1 was observed in African-American males, which exhibits a higher incidence of prostate cancer. These results indicate that downregulation of hZIP1 may be involved in the low zinc concentration observed in prostate cancer.

There is a recognized need in the art for methods of detecting and treating prostate cancer. Specifically, the prior art is deficient in the utilization of detectable biomarkers present in a prostate cancer. More specifically, the prior art is deficient in utilizing the human ZIP1 gene and the zinc and/or citrate levels regulated by expression of this gene for the detection and treatment of prostate cancer. The present invention fulfils this longstanding need in the art.

### SUMMARY OF THE INVENTION

The present invention is directed to a method of detecting prostate cancer. The method comprises determining an expression level of an hZIP1 polynucleotide in a prostate sample where a decrease in the expression level of the polynucleotide as compared to a control is indicative of prostate cancer. The hZIP1 polynucleotide may have the sequence shown in SEQ ID NO: 1. Determining the expression level may comprise detecting an hZIP1 polynucleotide in the prostate sample and in the control and comparing the amount of hZIP1 polynucleotide detected in the prostate sample to the amount of hZIP1 polynucleotide detected in the control. The level of hZIP1 detected correlates positively to the expression level of the hZIP1 polynucleotide. Alternatively, determining the expression level may comprise contacting the prostate sample and the control with an isolated antibody specific for an hZIP1 protein or a peptide fragment therefrom encoded by the hZIP1 polynucleotide of SEQ ID NO: 1 and comparing the amount of antibody bound to the hZIP1 protein or peptide fragment in the prostate sample to the amount of isolated antibody bound to the hZIP1 protein or a peptide fragment in the control. The amount of bound antibody correlates positively to the expression level of the hZIP1 polynucleotide in the prostate sample and control. The antibody may be specific for a hZIP1 protein having the sequence shown in SEQ ID NO: 4 or for a hZIP1 peptide fragment having the sequence shown in SEQ ID NO: 5.

The present invention also is directed to other methods of detecting prostate cancer. These methods comprise determining a zinc concentration or a citrate concentration in a prostate sample. A decrease in zinc concentration or in citrate concentration in the prostate sample as compared to a control zinc concentration or to a control citrate concentration is indicative of prostate cancer. A related method comprises detecting a second component in the sample. A concentration of the second component is the same in normal and cancerous prostate glands so that a decrease in the ratio of the concentration of zinc or a decrease in the ratio of the concentration of citrate in the prostate sample to the second component as compared to a ratio of zinc or citrate concentration to the second component in the control is indicative of prostate cancer.

The present invention is directed to a related method of detecting prostate cancer. The method comprises determining a zinc concentration and a citrate concentration in a sample. A decrease in the zinc and citrate concentrations as compared to a zinc concentration and a citrate concentration in a control is indicative of prostate cancer. A further related method comprises detecting a second component in the prostate sample, where the concentration of the second component in the prostate sample is the same in a normal and a cancerous prostate gland. A decrease in the ratio of the zinc concentration to the second component and a decrease in the ratio of the citrate concentration to the second component in the prostate sample as compared to the ratio of the zinc concentration to the second component and to the ratio of the citrate concentration in the control is indicative of prostate cancer.

The present invention is directed further to a method of treating prostate cancer. The method comprises administering an amount of a compound that increases expression of a hZIP1 gene in a prostate cell comprising the prostate cancer in an individual. A related method comprises a further method step of administering a second therapy selected from the group consisting of chemotherapy, radiotherapy, hormonal therapy, or cryosurgery to the individual, where the compound is administered before, during or after the second therapy is administered.

The present invention is directed further yet to an antibody directed specifically against a hZIP1 peptide or peptide fragment thereof comprising the sequence of SEQ ID NO: 4 or SEQ ID NO: 5. The present invention is directed further still to an expression vector comprising a genetic sequence effective to increase uptake of zinc into a prostate cell upon expression thereof.

The present invention is directed further still to a method increasing zinc uptake into a prostate cell. The method comprises contacting a prostate cell with the expression vector described herein in the presence of exogenous zinc. In a related method the prostate cell is a cancerous prostate cell and the method exhibits a first therapy against a prostate cancer such that a second therapy may be administered as described herein.

Other and further aspects, features, benefits, and advantages of the present invention will be apparent from the following description of the presently preferred embodiments of the invention given for the purpose of disclosure.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-1B show the nucleotide sequence of the hZIP1 polynucleotide (SEQ ID NO: 1; FIG. 1A) and amino acid sequence of hZIP1 protein (SEQ ID NO: 4; FIG. 1B). In FIG. 1A the underlined nucleotides 467 to 1428 encode the hZIP1 protein. In FIG. 1B the underlined amino acids 133-146 comprise the immunogenic peptide having SEQ ID NO: 5.

FIGS. 2A-2F show the immunohistochemical detection of ZIP1 transporter protein in malignant versus non-malignant glands. The transporter is localized to the plasma membrane. FIGS. 2A-2B show that the transporter is present in the normal and hypertrophic glands (which are also zinc-accumulating glands). FIGS. 2C-2D show that the hZIP1 is virtually absent (no plasma membrane staining) in the adenocarcinomatous glands and in prostatic intraepithelial neoplasia (PIN, thought by many to be a precursor stage of malignancy). FIGS. 2E-2F show the presence of hZIP1 in malignant prostate cell lines PC-3 and LNCaP respectively.

FIGS. 3A-3C show an in situ RT-PCR assay to detect ZIP1 mRNA in prostate tissue. FIG. 3A shows that adenocarcinomatous glands exhibit a complete absence of detectable ZIP) mRNA in the glandular epithelium (arrows). In contrast, in FIG. 3B shows the normal glands exhibit a high expression of ZIP1 (arrows), in the glandular epithelium; and no ZIP1 expression in the stroma. FIG. 3C shows the validation of the assay where GAPDH gene segment is amplified by the RT-in situ PCR method.

FIG. 4 shows a gel of the RT-PCR product of RNA extracted from malignant prostate tissue (PCa) and benign prostatatic hyperplasia (BPH). The gel clearly shows a marked decrease in ZIP1 mRNA in the malignant tissue. Density of the bands was determined by densitometry scans and GAPDH band intensity was used to normalize hZIP1 mRNA. h/ZIP1/GAPDH for PCa and BPH were 0.71±0.067 and 1.02±0.092, respectively.

FIGS. 5A-5F illustrate the zinc Levels in prostate sections. High zinc is represented by Newport Green yellow stain and low zinc is represented by TSQ red stain. The malignant region of the peripheral zone (FIG. 5A) shows a significant depletion of zinc in the malignant glandular epithelium as exhibited by the red staining (white arrows). The depletion of zinc is evident in early differentiated malignant glands as represented by combinations of red and yellow staining in the glandular epithelial cells. As malignancy advances to the undifferentiated stage, the zinc is further depleted as represented by the dominant red stain and no yellow stain in the glandular epithelium of the adenocarcinomatous glands (FIGS. 5A-5D). The depletion of zinc in the malignant glandular region results in the surrounding stroma showing a higher zinc level (green stain) than the glandular epithelium. In contrast, the normal peripheral zone glands (FIGS. 5E-5F) exhibit high zinc levels as represented by the uniform yellow stain and absence of red stain in the glandular epithelium. The stroma surrounding the glands exhibits a lower zinc level as shown by the red stain.

FIGS. 6A-6B illustrate the modified pyridine assay for citric acid. FIG. 6A shows the results obtained by performing the assay on sliced sections of resected rat ventral prostate glands. FIG. 6B shows a plot of the assay results.

FIGS. 7A-7B illustrate the energy dispersive X-Ray fluorescence detection of different components of prostatic tissue or fluid. FIG. 7A illustrates the energy dispersive X-Ray fluorescence detection of zinc, iron and copper. FIG. 7B shows the energy dispersive X-Ray fluorescence energy spectrum for filter disc spotted with increasing amounts of zinc.

### DETAILED DESCRIPTION OF THE INVENTION

### I. Definitions

As used herein, the term, “a” or “an” may mean one or more. As used herein in the claim(s), when used in conjunction with the word “comprising”, the words “a” or “an” may mean one but it is also consistent with the meaning of “one or more”, “at least one”, and “one or more than one”. Some embodiments of the invention may consist of or consist essentially of one or more elements, method steps, and/or methods of invention. It is contemplated that any method described herein can be implemented with respect to any other method described herein. As used herein “another” or “other” may mean at least a second or more of the same or different claim element or components thereof. As used herein “normal concentration or “normal level” or “control concentration” or “control level” indicates the concentration of a component that is present in a healthy non-cancerous sample. As used herein, the term “contacting” refers to any suitable method of bringing an inhibitory agent into contact with a human ZIP1 gene or polynucleotide thereof in a prostate cell or a prostate cancer or bringing an expression vector encoding a zinc-inducible promoter and hZIP1 gene or other gene effective to increase zinc uptake into prostate cells or prostate cancer cells. In vitro or ex vivo this is achieved by exposing the human ZIP1 gene or polynucleotide thereof or prostate cells or a prostate cancer comprising the same to the inhibitory agent in a suitable medium. For in vivo applications, any known method of administration is suitable as described herein. As used herein, the term “expression vector” refers to any plasmid, recombinant vector, viral vector, adenoviral vector or other vector having at least the minimum components to express a genetic sequence comprising the expression vector upon contact with a cell. As used herein, the terms “treating” or “treatment” includes prophylactic treatment as well as alleviation of ongoing or intermittent pathophysiological symptoms occurring in an individual with a prostate cancer. For example, treating a prostate cancer may restore zinc and/or citrate levels to those of a non-malignant or pre-malignant state. Thus, as would be obvious to one of ordinary skill, the term “individual” refers to any recipient of the treatment.

### II. Present Invention

In one embodiment of the present invention there is provided a method of detecting prostate cancer comprising determining an expression level of an hZIP1 polynucleotide in a prostate sample, where a decrease in the expression level of the polynucleotide as compared to a control is indicative of prostate cancer. In this embodiment the hZIP1 polynucleotide may have the sequence shown in SEQ ID NO: 1. In this and further embodiments, the prostate sample may be prostate tissue or prostatic fluid.

Further to this embodiment the method may comprise determining an expression level of the hZIP1 polynucleotide comprising amplifying hZIP1 polynucleotide mRNA from cells comprising the prostate sample and from the control sample; and comparing the amount of amplified hZIP1 cDNA in said prostate sample to that in the control sample wherein the level of hybridization positively correlates to the expression level of the hZIP1 polynucleotide. The hZIP1 polynucleotide may have the sequence shown in SEQ ID NO: 1

In another further embodiment the method may comprise contacting the prostate sample and the control sample with a antibody specific for hZIP1 protein or a peptide therefrom encoded by the hZIP1 polynucleotide; and comparing the amount of antibody bound to hZIP1 protein or a peptide therefrom in the prostate sample to that in the control sample wherein the amount of bound antibody positively correlates to the expression level of the hZIP1 polynucleotide. The antibody may be specific for a hZIP1 peptide having the sequence shown in SEQ ID NO: 2.

In another embodiment of the present invention there is provided a method of detecting prostate cancer comprising determining a zinc concentration in a prostate sample, where a decrease in zinc concentration in the sample as compared to a normal zinc concentration is indicative of prostate cancer. In this embodiment, the prostate sample may be prostate tissue or prostatic fluid.

In one aspect of this embodiment the zinc concentration may be determined by measuring zinc colorimetrically using 5-Br-PAPS [2-(5-bromo-2-pyridylazo)-5-(N-n-propyl.N-3-sulfopropylamino)phenol], 1-(2-pyridylazo)-2-naphthol or 4-(2-pyndylazo)resorcinol. Further to this aspect the method may comprise detecting a second component in the prostate sample, where a concentration of the second component is the same in normal and cancerous prostate glands so that a decrease in the ratio of the concentration of zinc to the second component as compared to a ratio in the control is indicative of prostate cancer. The second component may be copper, calcium, iron, or cadmium. In another aspect, zinc concentration may be determined by measuring zinc fluorometrically using energy dispersive X-Ray fluorescence. Further to this aspect the method may comprise detecting a second component in the prostate sample as described supra. In this aspect the second component may be copper, calcium, iron, or cadmium.

In yet another embodiment of the present invention there is provided a method of detecting prostate cancer comprising determining a citrate concentration in a prostate sample, wherein a decrease in the citrate concentration in the sample as compared to the citrate concentration in a control is indicative of prostate cancer.

In one aspect of this embodiment determining citrate concentration may comprise measuring citrate using a fluoroenzymatic assay. In another aspect determining citrate concentration may comprise measuring citrate using an acetic anhydride/pyridine assay having a sensitivity of at least 0.5 microgram of citrate. In yet another aspect determining citrate concentration may comprise measuring citrate using diazotized p-nitroaniline reagent and citrate lyase. In this further aspect citrate is converted to oxaloacetate. Further to this embodiment the method comprises detecting a second component in the prostate sample, where the concentration of the component is the same in normal and cancerous prostate glands so that a decrease in the ratio of the concentration of citrate to the component as compared to the normal ratio is indicative of prostate cancer. In an aspect of this further embodiment detecting lactate may comprise measuring lactate concentration using a fluoroenzymatic assay. In these embodiments, the prostate sample may be prostate tissue or prostatic fluid.

In a related embodiment the present invention provides a method of detecting prostate cancer comprising determining a zinc level and a citrate level in a prostate sample, where a decrease in the zinc and citrate levels as compared to a zinc level and a citrate level in a control is indicative of prostate cancer. In this embodiment the zinc level and the citrate level may be determined using the same or different methods. In one aspect determining the zinc level may comprise measuring the zinc level fluorometrically using energy dispersive X-Ray fluorescence detection.

Further to this embodiment the method comprises determining a level of a second component, where the level of the second component is the same in the prostate sample as the level in the control. In this further method step a decrease in the ratio of the zinc level to the level of the second component and a decrease in the ratio of the citrate level to the level of the second component is indicative of prostate cancer. In this further embodiment in determining the citrate level the second component may be lactate. Alternatively, in determining the zinc level the second component may be copper, calcium, iron, or cadmium. In these embodiments, the prostate sample may be prostate tissue or prostatic fluid.

In yet another embodiment of the present invention there is provided a method of treating prostate cancer, comprising inhibiting the downregulation of hZIP1 gene in a cell comprising the prostate cancer in an individual. Further to this embodiment the method comprises treating the individual with one or more other therapies effective against prostate cancer. Examples of other prostate cancer therapies are chemotherapy, radiotherapy, hormonal therapy, or cryosurgery.

In still another embodiment of the present invention there is provided an antibody directed specifically against a human ZIP1 peptide comprising SEQ ID NO: 2. In still another embodiment of the present invention there is provided an expression vector comprising a genetic sequence effective to increase uptake of zinc into a prostate cell upon expression thereof. An example of a genetic sequence is the sequence comprising SEQ ID NO: 1. In this embodiment the genetic sequence comprises a promoter inducible by exogenous zinc. Also, in this embodiment the expression vector may be a plasmid or a viral vector. An example of a viral vector is a plasmid or an adenoviral vector.

In still another embodiment of the present invention there is provided a method of increasing zinc uptake into a prostate cell, comprising contacting a prostate cell with the expression vector described supra in the presence of exogenous zinc. In an aspect of this embodiment the prostate cell is a cancerous prostate cell in an individual such that the method exhibits a therapeutic effect against a prostate cancer. Further to this aspect, the method may comprise administering a second therapy as described supra before, during or after administration of the expression vector.

The present invention provides methods of detecting prostate cancer using biomarkers and the subsequent treatment thereof. Prostate cancer may be detected by determining expression of the human ZIP1 (hZIP1) gene which regulates zinc uptake into prostate cells. Alternatively, determining the levels of zinc and/or citrate in the prostate tissue or prostatic fluid is a useful indicator of prostate cancer.

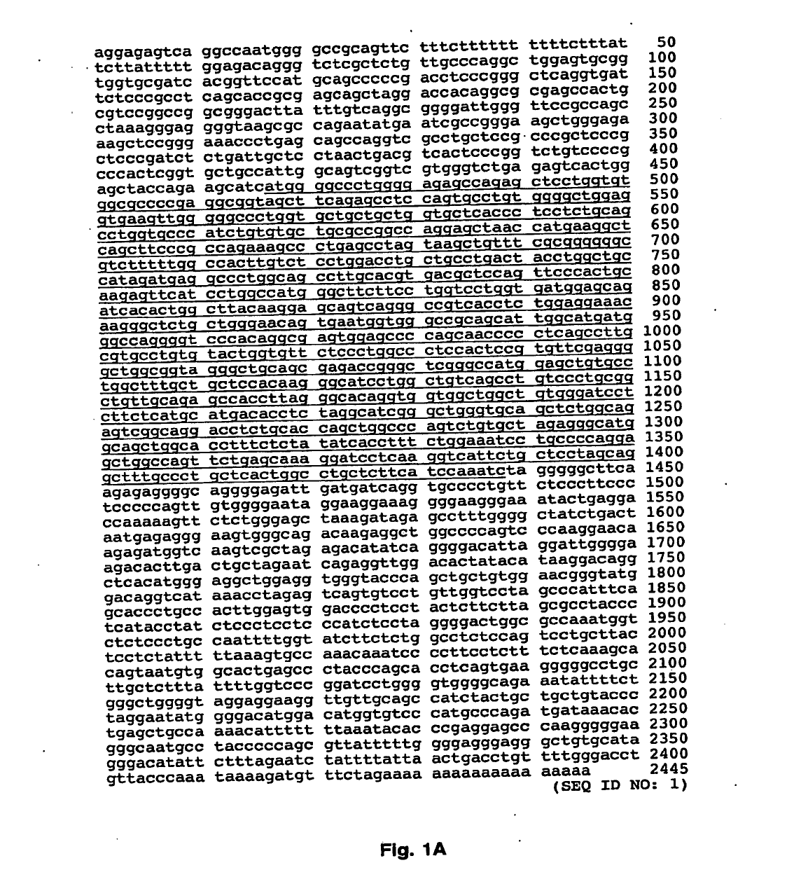

The present invention establishes hZIP1 downregulation in adenocarcinomatous prostate glands. Prostate cancer can be detected by examining the expression of hZIP1 gene or of a hZIP1 polynucleotide or of hZIP1 protein or peptide fragment encoded by the same. Downregulation of hZIP1 expression is indicative that the individual has prostate cancer. Preferably, the hZIP1 polynucleotide has the sequence shown in SEQ ID NO: 1 (FIG. 1A).

As is known and standard in the art, generally, hZIP1 expression can be quantified or determined at the nucleic acid level or at the protein level. At the nucleic acid level hZIP1 expression can be determined by a polymerase chain reaction (PCR), for example, Reverse Transcriptase-PCR (RT-PCR) using hZIP1 mRNA. At the protein level hZIP1 expression can be determined using an antibody directed against hZIP1 protein or a peptide therefrom by, for example, western blot analysis. The antibody may be raised against specifically against hZIP1 protein comprising SEQ ID NO: 4 (FIG. 1B). Preferably, the antibody is raised specifically against a hZIP1 peptide fragment comprising the amino acid sequence Tyr-Lys-Glu-Gln-Ser-Gly-Pro-Ser-Pro-Lys-Glu-Glu-Thr-Asn (SEQ ID NO: 5). Alternatively, Methods of raising antibodies specific for protein or peptide sequences are well-known and standard in the art (see, for example, Harlow et al. Cold Spring Harbor Laboratory Press 1988, Antibodies: A Laboratory Manual).

The present invention also provides methods of determining the cellular concentration of the biomarkers zinc and/or citrate as indicators of prostate cancer. A reduced level of zinc and/or citrate in prostatic fluid or prostate tissue indicates prostate cancer. Generally, prostatic fluid for assay purposes may be obtained during a digital rectal examination of the prostate. Prostate tissue sample is typically obtained via prostate biopsies using ultrasonic guidance.

A modified acetic anhydride/pyridine assay is provided to detect as little as 0.5 mg citrate in a prostate tissue or prostatic fluid sample. About 5 mg of tissue or 2 ml of prostatic fluid is required for this assay. The modified protocol is described in Example 8. Also, a modified oxaloacetic acid assay for detecting citrate in a prostatic tissue or fluid sample is provided. This method couples a citrate lyase reaction to detecting oxaloacetic acid using diazotized p-nitroaniline. The assay requires as little as 0.1 mg of tissue or 1 ml of prostatic fluid. This assay is about 10-100 fold more sensitive than the acetic anhydride/pyridine method. The modified protocol is described in Example 9. Furthermore, a modified fluorenzymatic citrate assay to measure citrate levels in prostatic tissue or fluid is provided. The modified protocol is described in Example 10. The amount of sample needed is about 50 mg of tissue or 1 ml of prostatic fluid. Multiple samples of ˜10-12/group run in triplicate can be assayed in about 30 minutes.

The present invention provides a colorimetric method of detecting prostate cancer in an individual by measuring the level of zinc in prostatic tissue or fluid. A low level of zinc as compared to a normal or control level is indicative of prostate cancer. The zinc in prostatic tissue or fluid may be determined using a colorimetric method with 5-Br-PAPS [2-(5-bromo-2-pyridylazo)-5-(N-n-propyl-N-3-sulfopropylamino)phenol], 1-(2-pyridylazo)-2-naphthol or 4-(2-pyridylazo)resorcinol. For example, if 5-Br-PAPS is employed, a visible red-pink chromogen (absorbance, 555 nm) is formed in the presence of zinc. The protocol is described in Example 11.

Alternatively, a method of determining zinc concentration in prostatic tissue or fluid using energy dispersive X-ray fluorescence is provided. A low concentration of zinc as compared to normal or control zinc concentration, i.e., ˜300-500 mg, is indicative of prostate cancer. The protocol is described in Example 12.

Energy dispersive X-ray fluorescence for the analysis of low concentrations of elements in biological samples eliminates many of the obstacles of sample preparation, ease of analysis and low concentration that restrict the application of sensitive methods such as Atomic Absorption Spectrometry (AAS) and other resource and time-intensive methods. In addition, energy dispersive X-ray fluorescence analysis does not result in destruction of the samples, thereby allowing re-analysis on the same samples. It is contemplated that a prostate sample is analyzed for zinc and then stored for subsequent analysis. Energy dispersive X-ray fluorescence analysis maybe used to measure the level of zinc in expressed prostatic fluid, semen, biopsy core samples and tissue sections. Prostatic fluid required for energy dispersive X-ray fluorescence analysis maybe collected on a filter disc during a digital rectal examination.

Energy dispersive X-ray fluorescence measurement of prostatic fluid and prostate tissue for zinc levels is a vast improvement over the current procedures for initial screening for prostate cancer. The “state of the art” today in energy dispersive X-ray fluorescence analysis is such that portable analyzers are commercially available. Energy dispersive X-ray fluorescence analysis is affordable and easy to perform. Thus, energy dispersive X-ray fluorescence analysis may easily integrate into a routine examination procedure carried out in a urologist and/or pathologist's facility.

One problem associated with employing absolute concentrations is the need to account for sample size. This limitation can be overcome if the specific compound being assayed is referenced to a constant component. As such, the present invention also provides a method of detecting prostate cancer by detecting and determining the concentration of a second component, e.g., copper, iron, calcium, or cadmium in determining zinc levels or lactate in determining citrate levels, in the prostate tissue or prostatic fluid sample. The concentration of the second component is the same in normal and cancerous prostate glands. Therefore, a decrease in the ratio of the concentration of zinc or of citrate to the second component as compared to a ratio in a control-normal sample is indicative of prostate cancer.

For example, the relatively simple determination of the citrate/lactate ratio in tissue samples using the fluoroenzymatic assay described in Example 10 provides an effective biomarker for detection of prostate cancer. This eliminates the need to consider and to determine the tissue weight or volume. The high concentrations of citrate and lactate in the normal tissue allows the fluoroenzymatic assays to be performed on biopsy cores, tissue sections, laser capture sections, and resected tissue.

It is contemplated that the downregulation of hZIP1 results in decreased zinc uptake by prostate cells. Decreased zinc uptake in turn decreases zinc's inhibitory effect on cell proliferation allowing for rapid proliferation of malignant prostate cells. If the downregulation of hZIP1 is inhibited then zinc uptake will increase and consequently rapid proliferation of malignant cells will decrease.

Thus, the present invention contemplates a method of treating prostate cancer. It is contemplated that a therapeutic benefit will be derived from inhibiting, preventing or reducing the down-regulation of human ZIP1 gene in malignant prostate tissue or cells. Concomittantly, zinc and/or citrate uptake is increased. Furthermore, this method may be used in combination with one or more other available treatments for prostate cancer, e.g., chemotherapy, hormone therapy, brachytherapy, radiotherapy and cryosurgery.

In addition, expression of the hZIP1 gene or polynucleotide therefrom or other gene effective to increase uptake of zinc may be induced in the presence of exogenous zinc. An expression vector comprising a promoter inducible in the presence of zinc and an hZIP1 DNA or polynucleotide or other gene upon contact with, introduction to or administration to a prostate cell may be effective to abrogate or inhibit hZIP1 downregulation, increase uptake of exogenous zinc or a combination thereof. Alternatively, hZIP1 cDNA or a polynucleotide therefrom, for example, including, but not limited to, hZIP1 DNA comprising SEQ ID NO: 1, may be inserted into a vector, such as a viral vector, suitable to deliver and to express the hZIP1 cDNA in a prostate cancer cell. These expression vectors or host cells comprising the same may be useful to increase hZIP1 expression in vitro or in vivo. In vivo administration of these vectors may be effective to exhibit a therapeutic effect against prostate cancer.

Methods of constructing expression vectors, e.g., plasmids or attenuated, replication-deficient viral vectors, e.g., adenoviral vectors, and their routes of administration are standard and well-known in the art. Current improvements in the art of viral vector construction, e.g., vaccinia viral vectors or adenoviral vectors, provide vectors designed to delete viral protein coding sequences so that cellular immune responses to viral-encoded proteins are at least largely diminished and so that large or multiple genes may be inserted into the viral genome.

### Pharmaceutical Compositions and Methods of Treating

Dosage formulations of a compound or a genetic sequence or a pharmaceutical composition thereof effective to inhibit downregulation of the human ZIP1 gene or to increase uptake of zinc also may comprise conventional non-toxic, physiologically or pharmaceutically acceptable carriers or other vehicles known and standard in the art suitable for the method of administration. For example, the compound, genetic sequence or pharmaceutical composition may be administered with a pharmaceutical carrier or may comprise a delivery vehicle, such as, but not limited to, a vector, a liposome, a nanosphere or microsphere, or a suitable gel or polymeric matrix. The compound, genetic sequence or pharmaceutical composition, vectors or delivery vehicles may be administered in combination with other anticancer therapy. In such a case, the anticancer drug of that therapy may be administered concurrently or sequentially with the compound, genetic sequence or pharmaceutical composition, vectors or delivery vehicles of the present invention. The effect of co-administration with an effective compound, genetic sequence or pharmaceutical composition, vectors or delivery vehicles is to lower the dosage of the anticancer drug normally required that is known to have at least a minimal pharmacological or therapeutic effect against a cancer or a cancer cell, for example, the dosage required to eliminate a cancer cell. Concomitantly, toxicity of the anticancer drug to normal cells, tissues and organs is reduced without reducing, ameliorating, eliminating or otherwise interfering with any cytotoxic, cytostatic, apoptotic or other killing or inhibitory therapeutic effect of the drug on cancer cells.

The compound, genetic sequence or pharmaceutical composition, vectors or delivery vehicles and anticancer drugs can be administered independently either systemically or locally, by any method standard in the art, for example, subcutaneously, intravenously, parenterally, intraperitoneally, intradermally, intramuscularly, topically, enterally, rectally, nasally, buccally, vaginally or by inhalation spray, by drug pump or contained within transdermal patch or an implant.

The compound, genetic sequence or pharmaceutical composition, vectors or delivery vehicles comprising the same or pharmaceutical compositions thereof may be administered independently one or more times to achieve, maintain or improve upon a therapeutic effect derived therefrom or to augment a therapeutic effect derived from other therapies suitable to treat the prostate cancer. It is well within the skill of an ordinary artisan to determine dosage or whether a suitable dosage comprises a single administered dose or multiple administered doses. An appropriate dosage depends on the subject's health, the progression or remission of the prostate cancer or the at risk status for prostate cancer, the route of administration, and the formulation used.

The following examples are given for the purpose of illustrating various embodiments of the invention and are not meant to limit the present invention in any fashion.

### Example 1

### Sample Collection

Expressed prostatic fluid is obtained during a digital rectal examination of the prostate. The rectal examination is done in the standard fashion. It is common for expressed prostate fluid to come out of the urethra during these exams and it can be collected on a slide, a small piece of paper or in a small vial. In some instances, extra palpation of the prostate is required to obtain secretions and in some cases, the patient needs to be instructed to relax his external sphincter and pelvic muscles to obtain fluid. One can generally obtain 0.25-1.5 ml of prostatic fluid during a typical digital rectal examination. The fluid sample can be refrigerated, frozen or processed according to the needs of the study.

Prostate biopsies are typically done through a transrectal approach using ultrasonic guidance. An ultrasound probe is placed transrectally, which enables the urologist to obtain both longitudinal and transverse images of the prostate. The advantage of an ultrasound guided prostate biopsy is that a specific area of the prostate can be biopsied under vision using ultrasound to guide the needle to the appropriate area. It is therefore possible to obtain biopsies from the peripheral and transition zones of the prostate. Typically, 12 core biopsies are taken during a prostate biopsy. Each core is 1-2.5 cm. in length. The risks associated with transrectal ultrasound of the prostate (TRUS) are discomfort from the ultrasound probe and/or biopsy needle, bleeding in the urine or stool or semen, and infection. To minimize these risks, Lidocaine is injected under ultrasound guidance around the prostate pedicle prior to biopsy to serve as a perineal block and minimize/eliminate pain from the biopsies. A quinolone antibiotic is typically given 1 day before and 3 days after the biopsy to minimize the risk of infection Specimens can be placed in formalin, frozen in liquid nitrogen or placed in other preservatives as needed by the study.

In some instances, men with bladder outlet obstruction from prostate enlargement require surgical resection of the tissue to open the bladder outlet, relieve symptoms and improve voiding function. There are several approaches that can be employed to obtain this goal, but the most frequently used approach is a transurethral resection of the prostate. During a typical transurethral resection of the prostate procedure, 10-100 grams of prostate tissue is obtained. This tissue can be preserved by any means well-known in the art.

### Example 2

### Immunohistochemistry of Human Prostate Tissue

Paraffin mounted serial sections of human prostate tissue was used for hZIP1 immunohistochemistry staining. Hematoxylin and eosin staining was used for identification of normal and adenocarcinomatous glands. The slides were dewaxed by incubation in xylene and then rehydrated. Non-specific binding of antibody was blocked by incubation in BLOKHEN (Ayes Labs, Inc) solution. The slides were washed with PBS, incubated in hZIP1 antibody solution, washed again, incubated with fluorescein-labeled secondary antibody solution, and then washed and mounted with anti-fade fluorescent medium (Molecular Probes). For control staining, adjacent serial sections were stained as described herein, except the antibody-depleted and preimmune preparation were used instead of anti-hZIP1 antibody.

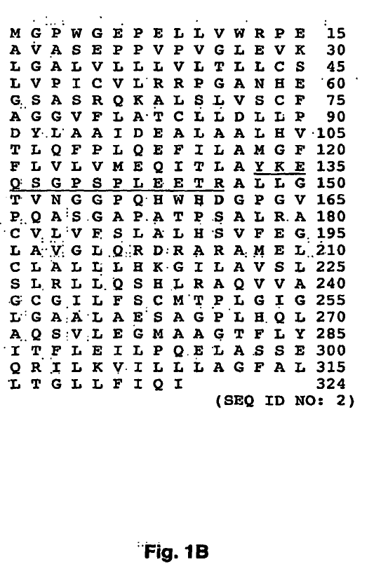

FIGS. 2A-2B show the membrane-associated immunohistochemical identification of hZIP1 in normal peripheral zone glandular epithelium. FIGS. 2C-2D show that hZIP1 is virtually absent in the adenocarcinomatous glands and in PIN, a precursor stage of malignancy.

### Example 3

### Immunocytochemistry of Prostate Cells

PC-3 and LNCaP cells were placed on cover slips. The cover slips were washed with PBS and the cells were fixed in paraformaldehyde solution. The cells were permeabilized by incubation in 0.2% NP-40 solution, were washed in PBS and were stained by the procedure described in Example 2.

FIGS. 2E-2F illustrate the presence of ZIP1 in malignant prostate cell lines PC-3 and LNCaP, respectively. The retention of ZIP1 gene expression in these malignant cell lines demonstrates that the absence of ZIP1 expression in the malignant glands in situ is not due to the deletion or fatal mutation of the gene. These results strongly implicate that epigenetic silencing of hZIP1 gene expression in the primary site malignant cells occurs in the in situ environmental conditions of the malignant prostate gland.

### Example 4

### RT-PCR of Human Tissue mRNA

hZIP1 and GAPDH cDNA were synthesized from total mRNA isolated from human prostate tissue using 1.0 mg of total RNA, reverse transcriptase and random primers. hZIP1 and GAPDH fragments were amplified from the cDNA using 1.0 mm forward and reverse primers and 35 cycles. The cloned cDNA for hZIP1 was used as the template DNA in control reactions to determine the specificity of the PCR reactions. The RT-PCR products were analyzed by agarose gel electrophoresis with ethidium bromide staining and photographed under UV light. The primers for hZIP1 were 5′-TCAGAGCCTCCAGTGCCTGT-3′ (SEQ ID NO: 2) and 5′-GCAGCAGGTCCAGGAGACAA-3′ (SEQ ID NO: 3).

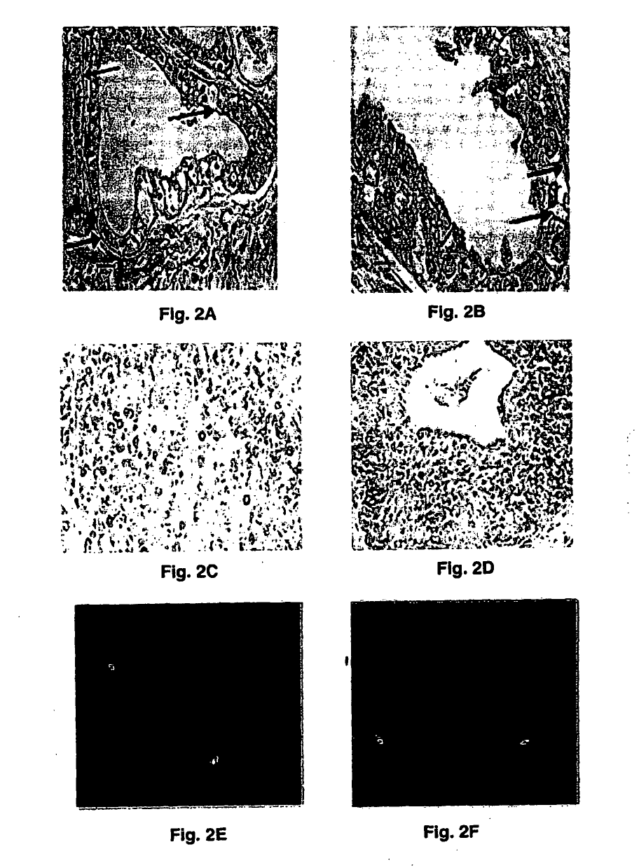

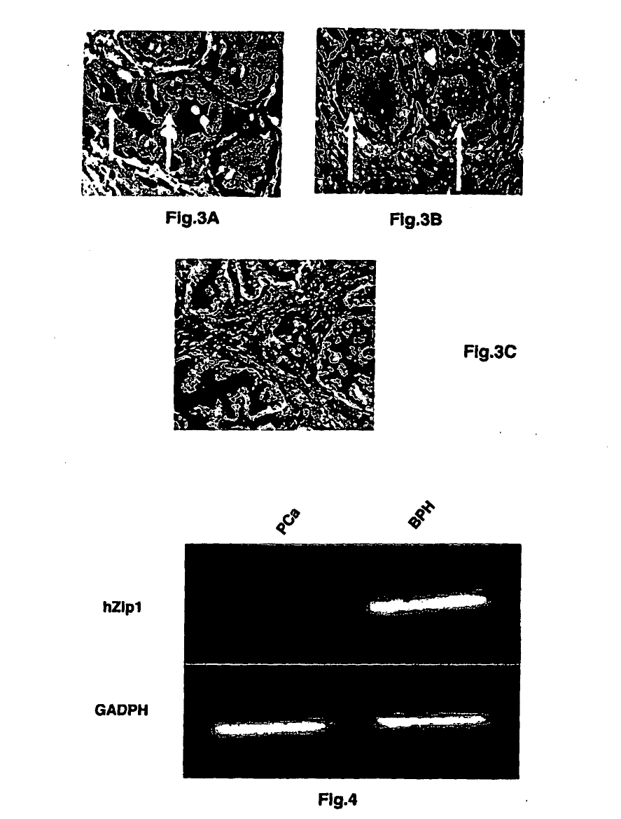

FIG. 3A shows a complete absence of detectable hZIP1 mRNA in glandular epithelium. In contrast, FIG. 3B shows that normal glands exhibit a high expression of ZIP1 in the glandular epithelium. FIG. 4 shows the RT-PCR analysis of ZIP1 expression in tissue extracts of malignant tissue versus benign hyperplastic glands, which like normal peripheral zone, are zinc-accumulating glands. The decrease in ZIP1 mRNA in the cancer tissue is clear from this gel profile.

### Example 5

### Determination of Intracellular Zinc Content

The relative intracellular zinc content in situ was determined by utilizing fresh frozen tissues. For this purpose, the cells must be biochemically active. The relative concentrations of zinc in various cell types of prostatic tissues were determined according to the manufacturer's protocol. The frozen tissues were incubated with equal molar concentrations of two zinc-indicator dyes, i.e., Newport Green (NPG) and TSQ. The frozen tissues were incubated in 20 ml/section of the zinc indicator cocktail overnight and washed gently in PBS without disturbing the tissues. The slides were heat fixed for 10 sec at 104° C. to immobilize the signals. These slides were mounted with solution containing 50% glycerol in PBS and observed under a fluorescence microscope.

TSQ has a high affinity for zinc (Kd˜10 nM) and a detection limit of ˜0.1 nM. The Zn-TSQ positive cells stain red. NPG has moderate zinc-binding affinity (Kd˜1 mM). The Zn-NPG positive cells appear yellowish green. Together, TSQ and NPG provide a relative difference in zinc concentrations in various cell types of the prostate. TSQ provides about 2-3 log higher affinity for zinc as compared to NPG, but provides a detection limit of about 3-log lower than NPG. Therefore, cells that contain very low concentrations of intracellular zinc appear red and the cells with higher concentrations appear green. The cells with no detectable zinc will appear black or dark blue.

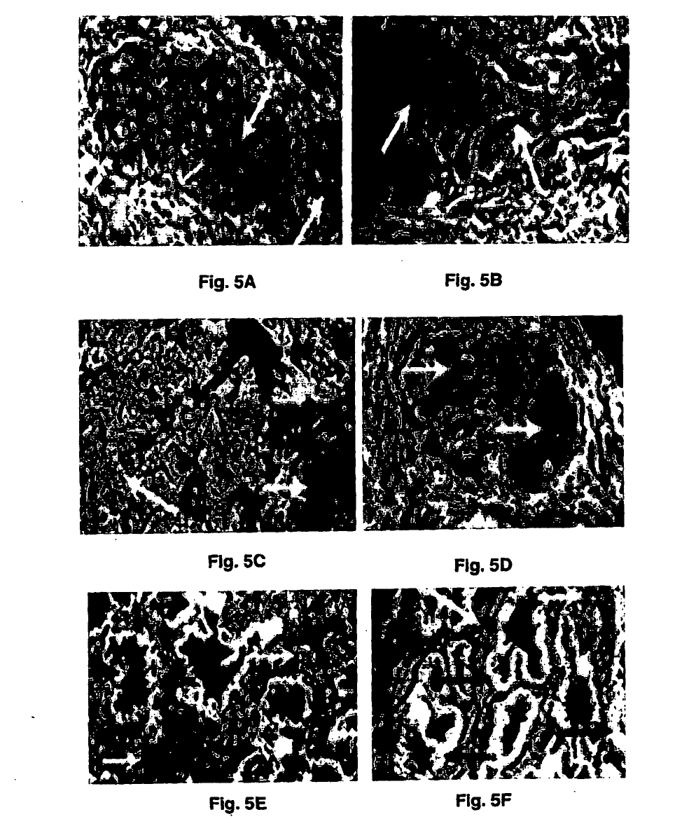

High zinc is represented by Newport Green yellow stain and low zinc is represented by TSQ red stain. The malignant region of the peripheral zone in FIG. 5A shows a significant depletion of zinc in the malignant glandular epithelium as exhibited by the red staining (white arrows). The depletion of zinc is evident in early differentiated malignant glands as represented by combinations of red and yellow staining in the glandular epithelial cells. As malignancy advances to the undifferentiated stage, the zinc is further depleted, as represented by the dominant red stain and no yellow stain in the glandular epithelium of the adenocarcinomatous glands in FIGS. 5A-5D. The depletion of zinc in the malignant glandular region results in the surrounding stroma showing a higher zinc level (green stain) than the glandular epithelium. In contrast, the normal peripheral zone glands in FIGS. 5E-5F exhibit high zinc levels as represented by the uniform yellow stain and absence of red stain in the glandular epithelium. The stroma surrounding the glands exhibits a lower zinc level as shown by the red stain.

### Example 6

### Refinement Assays

The zinc and citrate assays are modified by first assessing the feasibility of such modifications on prostate tissues of rats. For each assay a sample size of 6 rats in each group, i.e., cancerous and normal/benign, is used.

### Example 7

### Feasibility Study of the Prostate Cancer Biomarker

The feasibility study of using zinc, citrate and hZIP1 expression as prostate cancer biomarkers is based on the Receiver Operating Characteristics (ROC) methodology (9). The ROC method now is used commonly to evaluate the diagnostic performance of biomarkers. ROC displays the relationship between sensitivity and specificity across all cut points of the test.

The Area Under the ROC curve (AUROC) measurement is a measure of the predictive power of the marker of the disease. In the instant invention, the AUROC measurement for cancerous vs. normal issues is used to determine the discriminative accuracy of zinc and citrate. As discussed herein, the specificity of the PSA-based method used currently for detecting prostate cancer is plagued with false positives in 37% of cases. The specificity of the current method is only 67%, thereby subjecting a larger portion of patients to unnecessary biopsies. The sensitivity of the PSA method is above 90% while the AUROC is less than 0.80.

It is contemplated that AUROCs of the citrate and zinc markers are at least 0.90. The sample size needed to achieve an AUROC of at least 0.8 at a significance level 0.025 with 90% power (at AUROC=0.90) is 86 samples in each category, i.e., cancerous or normal. To account for an estimated 10% non-evaluable samples, 95 samples with one sampler per patient in each category is sufficient.

Continuous markers' values are dichotomized into “positive” and “negative,” using ROC curve analysis to select cutoff points for zinc and citrate. Once cutoffs have been established, the sensitivity and specificity of each dichotomized marker is calculated using 2×2 contingency tables. In addition, the maximal chi-squared method of Miller and Siegmund (10) can be used to determine which threshold of each marker best classifies patients into cancer and non-cancer subgroups.

To reduce potential overall type I error, the search is focused on the potentially most predictive ranges of values and a Bonferroni corrected p-value is used as an index of the strength of the prediction (11). 95% confidence intervals for the true sensitivity and specificity of the method relative to biopsy are estimated. Positive and negative predictive values are estimated using the standard Bayesian formula. Further exploration of the markers' ability to predict prostate cancer can be conducted using a logistic regression analysis, including that of a 3-class model with cancer, no cancer and questionable, where the probability of prostate cancer given the level(s) of the marker(s), the possible combinations of the markers and other patient characteristics such as age can be modeled. The predictive power of the 3-class cancer model can be evaluated again using the ROC method in Reik et al. (12). Cross-validation errors can be calculated by deleting one sample and 25% of the samples. This statistical method predicts the sensitivity and specificity of the markers' ability to detect cancer using a three-class cancer model, i.e., includes the three possible outcomes of cancer, no cancer or questionable.

### Example 8

### Colorimetric Citric Acid Assay (Pyridine Method)

The acetic anhydride/pyridine method is successfully modified as a micro-method providing detection at a level of about 0.5 mg of citrate. A 5% TCA extract of the tissue/prostatic fluid sample is prepared and 20 ml of the TCA extract is placed in a 1.5 ml microfuge tube. 160 ml of acetic anhydride is added, the tube is capped, shaken briefly and placed in a 60° C. water bath for 10 min; and centrifuge for 2 min. The supernatant is transferred to a new microfuge tube whereupon 20 ml pyridine is added, the tube is capped and shaken briefly and placed in water bath for 30 min. The yellow color of the sample is compared, visually or with a photometer at 425 nm, to the color of the sample with 2-3 standards and a blank that are run simultaneously with the sample. Tissue samples should be rapidly frozen or placed in 5% TCA until the assay is performed.

For human prostate tissue, samples representing prostate cancer will contain ˜10% or less of the citric acid found in normal prostate samples or BPH (benign prostate hyperplasia) samples. The following are some estimated values using the above protocol. A 1 mg wet weight sample of normal prostate tissue sample contains about 2 mg citric acid, which is at the minimal detection level of the assay. For reliability, about 5 mg of sample is preferred. This would accommodate tissue sections, resected tissue samples, laser capture samples, but possibly might exclude single biopsy cores.

Samples of non-malignant prostate tissue give a highly visible yellow color. In contrast, under the same conditions, a malignant tissue sample would contain about 0.2 mg citric acid in the reaction, which will produce a barely discernible color development above the blank. Included in the assay are the blank (no citrate) and standards of known citrate concentration. The minimal citrate concentration above which would reflect >80% accuracy for non-malignant sample, i.e., negative for prostate cancer, and the maximal citrate concentration below which would represent >80% accuracy for a malignant sample for prostate cancer are represented in the standards. In between these ranges, there is a range of values that constitutes inconclusive results.

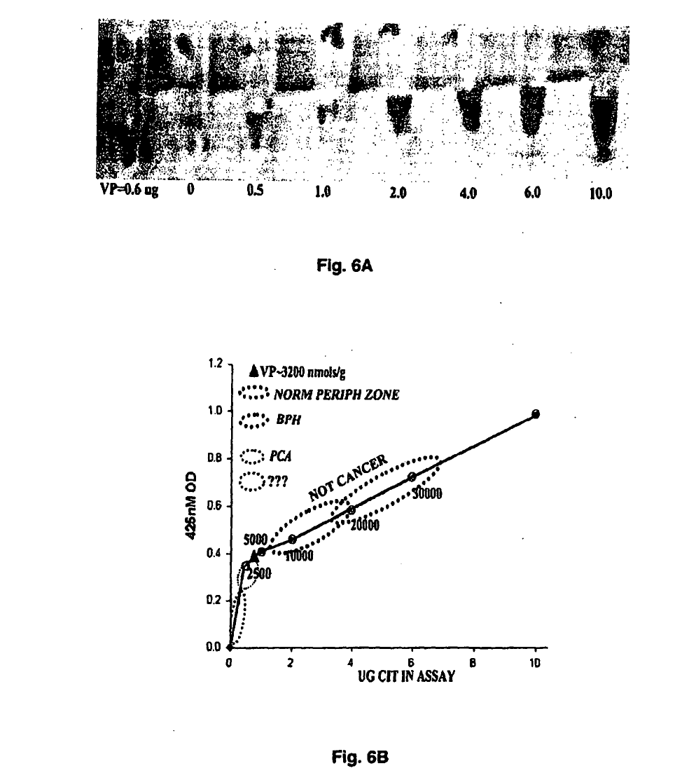

The assay was performed on resected rat ventral prostate glands. The prostate glands were sliced into sections weighing ˜1 mg for the assay. The assay was performed as described above and depicted in FIG. 6A. The color development was measured in a photometer at 425 nm. The results are plotted in FIG. 6B. The range represented by ˜1 mg samples of malignant tissue, normal peripheral zone and BPH is shown. The accuracy of the assay also is evident from the ˜3000 nmols/gram value obtained from a rat ventral prostate without added citrate. The value would be at the high limit of the range for a malignant prostate tissue sample.

Normal prostatic fluid contains ˜90 mM citrate with a range of ˜50-130 mM. This translates to ˜90 umols/ml (˜18,000 ug/ml or ˜18 ug/ul). Therefore, 1 ml of prostatic fluid is sufficient for the determination of citrate. Prostate cancer prostatic fluid contains citrate at a concentration that is ˜1-10% of the normal prostatic fluid, i.e., 1 ml contains ˜2 mg citrate. With these estimates, the citrate assay can be performed with as little as ˜1-2 ml of prostatic fluid. For most males undergoing digital rectal examination, prostatic massage will produce 10-50 ml of expressed prostatic fluid. The prostatic fluid is extracted with 5% TCA for the citrate assay.

### Example 9

### Colorimetric Assay for Citrate (Nitroaniline/Oxalo Acetic Acid Method)

The pyridine method in Example 8 is applicable to many different samples where 1 mg or more of sample is available. The sensitive colorimetric tests for oxalacetic acid using diazotized p-nitroaniline has a linear range of 0.003-0.03 mmole in a 7 ml assay volume which translates to 0.0002-0.0002 mmol/ml sensitivity range. This assay can be modified to detect citrate by using citrate lyase, which converts citrate to oxaloacetate. At this sensitivity the assay detects at least about 0.04 mg citrate in a 1 ml assay volume or 0.008 mg citrate in a 200 ml volume. At this sensitivity, i.e., ˜10-100 fold more sensitive than the pyridine method, citrate is assayed in prostate samples of about 0.1 mg.

To adapt this method for citrate in prostate samples, the protocol utilizes the reaction: Citrate+Citrate Lyase→oxaloacetic acid+acetate. A prostate tissue/prostatic fluid sample is vortexed in 100 ml 10 mM Hepes buffer at pH˜7.4 and citrate lyase is added for 15 minutes to catalyze the above reaction. 3.5 ml 70% TCA is added to the sample and the sample is vortexed and centrifuged. The supernatent is transferred to a microfuge. 100 ml of acetate buffer, pH˜5.2, is added followed by 50 ml of diazotized p-nitroaniline reagent. Yellow chromogen is visualized or read in a photometer (455 nm).

### Example 10

### Fluoroenzymatic Citrate Assay

The major advantages of the colorimetric methods described above are their rapidity and simplicity to perform and the capability to employ as a visual test, if so desired. The drawback is the sensitivity/detection level that can impose problems for limited sample size, such as biopsy material. This drawback is eliminated by the application of the fluoroenzymatic assay of citrate.

The assay reaction for fluorescence detection of citrate is:

(1) Citrate+Citrate Lyase→Oxaloacetate+Acetate

(2) Oxaloacetate+NADH+H⁺L-MDH→L-malate+NAD⁺

The oxidation of NADH to NAD is determined with a fluorometer under the conditions described initially by Lowry et al. (13). The assay has a sensitivity down to ˜10⁻⁷ M (0.1 mM); which is ˜10⁶ times lower than the citrate concentration of normal prostatic tissue. Under these conditions the citrate is detected in <mg tissue samples; so that all types of tissue samples, including biopsy cores or sections of biopsy cores, will be assayable.

To perform the assay, 900 ml of 100 mM Tricine buffer (pH=8.2) containing 1.0 mM MgCl₂ and citrate lyase enzyme is added to a fluorometer tube and fluorescence is zeroed using a blank. 10 ml of an appropriate concentration of NADH (stock NADH conc is determined photometrically) is added and the initial fluorescence (Fi) is determined. 1 ml of malate dehydrogenase (MDH) is added to allow the reaction to proceed to completion over 10 min and the fluorescence (Fr) is determined. 1 ml of excess oxaloacetic acid is added and the final fluorescence (Ff) is determined.

The citrate concentration is determined by: Fi minus Ff=FNADH; the fluorescence due to the total concentration of NADH; and Fi minus Fr=FCitrate; the fluorescence due to the citrate in the sample. The ratio of FCitrate/FNADH×conc NADH=concentration of citrate in the assay, which is multiplied by the sample dilution to obtain the citrate concentration of the original tissue sample.

The assay of a single sample run in triplicate can be performed in about 15 minutes. Multiple samples of ˜10-12/group run in triplicate, i.e., ˜30-36 reaction tubes, can be performed in about 30 minutes. The fluorometer can interface with a computer so that all fluorometer readings and sample information are entered directly into the computer to store the data and to calculate the results. The assay procedure is simple to perform.

For a section of a biopsy core that might provide 50 mg of sample, if this biopsy is from the normal peripheral zone, then the 50 mg sample will contain ˜0.5 mmol citrate. The tissue can be placed in 5% TCA for deproteinization, for example, 100 ml of TCA. 10 ml aliquots, generally with 3 replicates, of the TCA extract can be used for the assay. This produces a citrate concentration in the assay reaction tube of ˜5×10⁻⁵ M. If the sample is malignant, the citrate content would be ˜0.05 μmol, which would be ˜0.05×10⁻⁶ M. These are ranges of citrate that are easily and accurately measured by the fluorometric assay. In most instances, the tissue availability for assay will be greater than represented in this example. If the fluoroenzymatic assay is employed for prostatic fluid which contains ˜90 mM citrate one could assay as little as 1 ml sample volumes.

The operational sensitivity limit of the spectrophotometric assay of NADH/NADPH is generally around 10⁻⁴ M, possibly 5×10⁻⁵ M. Therefore, it will not be suitable for biopsy cores and other limited amounts of sample, especially involving replicate determinations. Also, the range is limited by the absorbance measurements and becomes cumbersome for samples that vary over a magnitude range. Because the measurements are in the UV range, the cuvettes impose some limitations. None of these drawbacks exist with the fluoroenzymatic assay. It is ˜100-1000-times more sensitive than photometry. Disposable, low cost fluorometer tubes (10×75 culture tubes) can be

| | Lactate† | Citrate† | LA/CA Ratio |
|---|---|---|---|
| BPH, 19 | 6.15 (3.91-9.62) | 10.20 (6.11-16.65) | 0.603 (0.32-0.64) |
| Early | 6.50 (2.37-11.40) | 6.60 (3.13-11.49) | 0.985 (0.76-1.55) |
| PCA, 19 | | | |
| Advanced | 4.76 (3.14-7.62) | 1.40 (0.49-2.72) | 3.40 (2.80-6.40) |
| PCA, 10 | | | |

employed for these assays. Multiple tubes can be serially assayed within minutes. The assay is best conducted with a simple filter fluorometer, which is relatively inexpensive.

The methods described in the above Examples require that the weight or volume of prostate sample be determined so that the relative concentrations of citrate or zinc can be determined. An alternative that eliminates the need for sample size is the “referencing” method in which changes in citrate will be determined with a second factor that does not change. This can be achieved by the citrate/lactate ratio based on the study of Cooper and Farid (14) as shown in Table 1. They reported that citrate decreased in prostate cancer tissue but lactate remained constant. Thus, the citrate/lactate ratio could be an index of prostate malignancy.

TABLE 1
The citrate/lactate ratio

| | Lactate | Citrate | LA/CA Ratio |
|---|---|---|---|
| BPH, 19 | 6.15 (3.91-9.62) | 10.20 (6.11-16.6) | 0.603 (0.32-0.64) |
| Early | 6.50 (2.37-11.40) | 6.60 (3.13-11.49) | 0.985 (0.76-1.55) |
| PCA, 19 | | | |
| Advanced | 4.76 (3.14-7.62) | 1.40 (0.49-2.72) | 3.40 (2.80-6.40) |
| PCA | | | |

This early study involved chromatographic determination of citrate and lactate and required a large amount of tissue, e.g., 100-200 mg of combined biopsy tissue and >1 gram of resected prostate tissue. The large samples introduce considerable variability in that considerable mixing of peripheral zone and central zone likely occurs. In addition, the transformation of the chromatographic peaks to absolute concentrations of the specific substrate inherently introduces variability. The application of fluoroenzymatic assays with high sensitivity, coupled with much smaller and more homogeneous peripheral zone samples, provides minimal variability in the precision of the concentrations of citrate and lactate. A small tissue sample that “focuses” the region of expected or suspected primary malignant site would reveal much greater differences than reflected in this report.

For lactate the fluoroenzymatic assay is based on the reaction

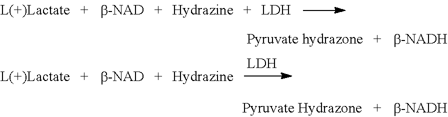

whereby hydrazine traps pyruvate to force the reaction to completion.

### Example 11

### Colorimetric Assay for Zinc

Several colorimetric assays for zinc in blood and other biological fluids, e.g., urine and cerebrospinal fluid, currently exist. These assays were developed mainly, but not solely, for the measurement of zinc in blood plasma, which is ˜1 mg/ml (˜0.015 mM), and they operate generally over the range of ˜0.01-2.0 mg/ml, i.e., the concentrations in the assay system. Tissue, excluding prostate, zinc levels are ˜0.2 mM and, therefore, should be readily detected by modification of these assays. Moreover, normal peripheral zone zinc levels are ˜3 mM and normal prostatic fluid zinc is ˜9 mM. It is contemplated that the colorimetric determination of zinc in prostate samples is readily achievable. It is contemplated that the colorimetric methods utilizing 5-Br-PAPS or [2-(5-bromo-2-pyridylazo)-5-(N-n-propyl-N-3-sulfopropylamino)phenol] (15), 1-(2-pyridylazo)-2-naphthol (16) or 4-(2-pyndylazo)resorcinol (17) can be modified and adapted for the measurement of zinc in prostate tissue and prostatic fluid samples.

For example, one can use the 5-Br-PAPS method for detection of zinc in prostate tissue. A sample of normal prostate tissue weighing 1 mg will contain ˜0.2 mg zinc, which is well above the assay detection limit of ˜0.01 mg/ml. When 5-Br-PAPS is utilized, a clearly visible red-pink chromogen (absorbance, 555 nm) is produced in the presence of zinc. A prostate cancer tissue sample of 1 mg will contain ˜0.02 mg of zinc which will be barely detectable in this assay. The assay will include standards for the determination of the tissue zinc concentration. If necessary, the assay volume can be decreased to, for example, 0.5 ml which would correspondingly increase the assay concentration of zinc.

### Example 12

### Energy Dispersive X-Ray Fluorescence (EDXRF) for Zinc Analysis in Prostate Samples

Energy dispersive X-ray fluorescence for the analysis of low concentrations of elements in biological samples eliminates many of the obstacles of sample preparation, ease of analysis and low concentration that restrict the application of sensitive methods such as Atomic Absorption Spectrometry and other tedious methods. In addition, energy dispersive X-ray fluorescence analysis does not result in destruction of the samples, so that re-analysis and other assays are possible on the same samples. Indeed, one can envision that a subject's prostate sample could be analyzed for zinc and then stored for follow-up examination for comparison. The energy dispersive X-ray fluorescence application is used to measure the level of zinc in expressed prostatic fluid, semen, biopsy core samples and tissue sections.

The studies of Zaichick at al. (6) with prostatic fluid samples and prostate tissue samples provide the major supporting background for this zinc detection method. Hall (18) successfully analyzed zinc in 10 mg prostate tissue sections with X-ray fluorescence. Vartsky et al. (19) recently applied energy dispersive X-ray fluorescence for the detection of zinc levels in surgically resected prostate tissue samples.

In initial studies the analysis of small volumes of fluids containing Zn were characterized. Based on earlier reports, expected concentration of Zn in normal prostatic fluid is ˜300-500 mg/ml. In addition the volume of prostatic fluid collected by DRE (prostate massage) minimally will be ˜200 ml, which will contain ˜30-50 mg zinc that is easily detected by energy dispersive X-ray fluorescence. Correspondingly, a 200 ml sample from prostate cancer would contain ˜3-5 mg zinc, which is also detectable by energy dispersive X-ray fluorescence. Once collected, the sample or an aliquot can be directly measured.

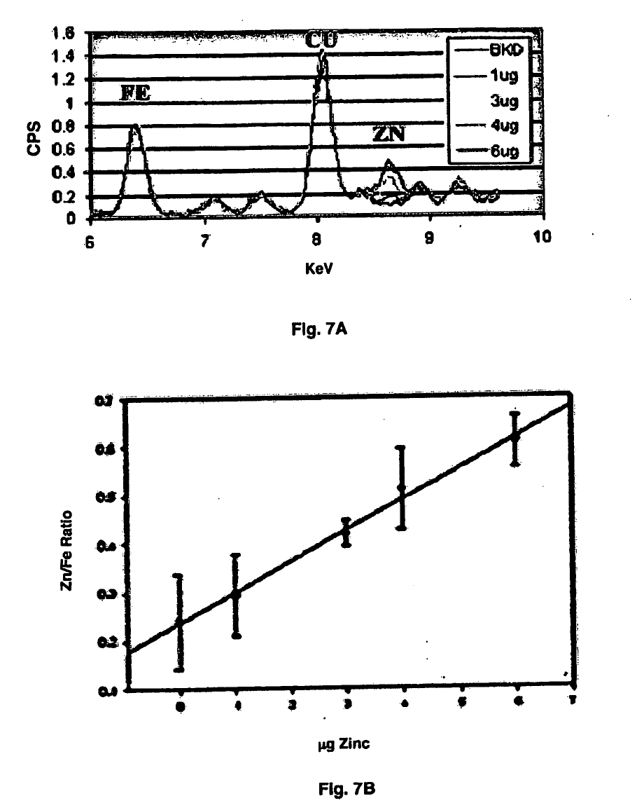

One procedure employed is to collect expressed prostatic fluid on filter paper disks. These disks can be the samples of fluid that is analyzed. To characterize the analysis, the total amount of zinc in a “standardized” area on the disk is determined. A series of Zn standards containing various amounts of Zn (1-50 mg) in various volumes (10-40 ml) can be used. The concentrations of the zinc standards may be determined by atomic absorption spectroscopy. Standards are spotted onto filter paper disks identical to those used to collect prostatic fluids. All samples can be spotted in triplicate and analyzed. FIG. 7A illustrates the energy dispersive. X-ray fluorescence detection of zinc, iron and copper. FIG. 7B shows the energy dispersive X-ray fluorescence energy spectrum for filter disc spotted with increasing amounts of zinc. A standard curve for different amounts of zinc spotted on filter paper disks determined by energy dispersive X-ray fluorescence is used.

Energy dispersive X-ray fluorescence analysis of prostatic fluid from rat ventral and lateral prostate can be assayed to validate the measurements on actual biological fluid. Rat prostate tissue and prostate fluid contain high levels of zinc like human prostate. In addition, the level of zinc in the rat prostate is regulated by androgens; therefore, castration results in a decrease in the zinc content of the prostate and its fluid in a physiological context.

For these assays lateral and ventral prostate fluid from intact and castrate rats is collected. The fluid samples are spotted on filter paper disk. An aliquot of fluid is extracted and analyzed by AAS to determine the zinc concentration. The filter disks spotted with fluid is analyzed by EDXRF. The X-ray energy spectrum obtained from the analysis is used to calculate the Zn/X ratio, where X is an element present in prostatic fluid that is independent of prostate status. These elements can be identified by comparing their levels in fluid from normal and castrate rats. The Zn/Fe ratio is used, however, other elements, e.g. Cu, also can be considered.

In an alternative approach a fixed area of the filter paper disks is analyzed. This ensures near equal volume. To achieve this a “standardized” circular area of the fluid spot is punched out from the filter for analysis. The zinc content is compared with the concentration from AAS or colorimetric analysis to validate the results of the assay.

To determine the sensitivity of the assay, a mock prostatic fluid containing all of the component salts and elements of normal prostatic fluid except zinc is used. Bovine serum albumin. is added to simulate the protein components. Increasing concentrations of zinc is added to the mock fluid in 0.5 ml/ml increments. Equal volumes (10 μl) of mock prostatic fluid are spotted on filter disks in triplicate and are assayed by EDXRF. Results from the EDXRF analysis are plotted against the actual zinc concentrations determined by AAS. From these result the limits of detection by EDXRF and thereby the sensitivity of the EDXRF assay is determined.

To perform EDXRF on prostatic fluid samples, these samples will be collected as a part of routine urological examinations. The prostatic fluid is collected during a digital rectal examination on filter paper disks specifically designed for collecting fluid. Typically the digital rectal examination results in the collection of 10-30 ml of fluid. The filter paper disks are analyzed directly with no further sample preparation needed by EDXRF.

This technique is beneficial because of its ease of operation as an “office/examining room” procedure. The urologist is able to perform the prostate massage and to collect the prostatic fluid directly onto the filter disc, as part of the digital rectal exam. The “standard” punch of the filter disc is obtained and can be analyzed immediately by a “portable” EDXRF instrument in the examining suite. Within minutes the zinc value or zinc ratio, e.g., Zn/Fe, is obtained. Comparison of the zinc value with established ranges for cancer and non-cancer provides information for the urologist's diagnostic examination. The filter disc can be retained with the subject's records and compared with future samples during follow-up examinations or can be used for a citrate analysis, as described in Example 8, for confirmational information on the same sample.

A major advantage of the EDXRF analysis is that samples are not destroyed and thus the same samples can be used in other assays. In addition, tissues that have been fixed can also be subjected to EDXRF analysis. To apply EDXRF to tissues, tissue sections are fixed on glass slides. Paraffin embedded and frozen sections of rat prostate tissue can be prepared for EDXRF analysis. Rat lateral prostate tissue for intact and castrate animals, both frozen and paraffin imbedded, are sectioned at various thicknesses. Adjacent pieces of tissue are collected and are extracted for AAS analysis. The tissue section is processed and is mounted on glass slides by standard methods. Slides are analyzed by EDXRF to determine the zinc content of sections. A similar approach can be used to apply the assay to human prostate tissue sections.

The following references are cited herein:
1. Zakian et al. (2003) Radiology, 229:241-247.
2. Kurhanewicz et al. (1996) Radiology, 198:795-805.
3. Costello et al. (1999) J Biol Chem 274:17499-17504.
4. Costello et al. (2002) The metabolic characterization of prostate malignancy by magnetic resonance spectroscopy. In: Encyclopedia of Cancer, Academic Press.
5. Kurhanewicz et al. (2002) J Magn Reson Imaging, 16(4):451-63
6. Zaichick et al. (1997) Int Urol Nephr 29:565-574.
7. Franklin et al. (2003) J Inorgan Biochem 96:435-442.
8. Franklin et al. (2005) Mol Cancer, 4:32.
9. Zhou et al. (2002) Statistical Methods in Diagnostic Medicine. John Wiley & Sons, New York.
10. Miller R and Siegmund D. (1982) Biometrics 38:1017-1023.
11. Hilsenbeck and Clark, (1996) Statistics in Medicine, 15(1):103-12.
12. Reik et al. (1995) Journal of Theoretical Biology, 172:245-258.
13. Lowry et al. (1951).
14. Cooper, J E and Farid, I (1964) J Urol. 92:533-536.
15. Homsher, R. and Zak, B. (1985) Clin Chem., 31(8):1310-3
16. Fuentes et al., (1982) Andrologia, 14(4):322-7.
17. Lampugnani, L and Maccheroni, (1984) M. Clin Chem., 30(8):1366-8.
18. Hall, T. (1961) Science, 134:449-55.
19. Vartsky et al, (2003) J Urol. 170(6 Pt 1):2258-62.

Any publications or patents mentioned in this specification are indicative of the levels of those skilled in the art to which the invention pertains. Further, these publications are incorporated by reference herein to the same extent as if each individual publication was specifically and individually incorporated by reference.

One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method of detecting prostate cancer comprising:
determining an expression level of an hZIP1 polynucleotide in a prostate sample, wherein a decrease in the expression level of said polynucleotide in said prostate sample, as compared to a control is indicative of prostate cancer.

2. The method of claim 1, wherein determining an expression level of the hZIP1 polynucleotide comprises:
detecting an hZIP1 polynucleotide in the prostate sample and in the control; and
comparing the amount of hZIP1 polynucleotide in said prostate sample to the amount of hZIP1 polynucleotide in the control, wherein the level of hZIP1 detected correlates positively to the expression level of the hZIP1 polynucleotide.

3. The method of claim 2, wherein said detecting step comprises amplifying the hZIP1 polynucleotide.

4. The method of claim 2, wherein said hZIP1 polynucleotide comprises the sequence of SEQ ID NO: 1.

5. The method of claim 1, wherein determining an expression level of the hZIP1 polynucleotide comprises:
contacting the prostate sample and the control with an isolated antibody specific for an hZIP1 protein or a peptide fragment therefrom encoded by the hZIP1 polynucleotide of SEQ ID NO: 1; and
comparing the amount of antibody bound to said hZIP1 protein or peptide fragment in the prostate sample to the amount of isolated antibody bound to the hZIP1 protein or a peptide fragment in the control wherein the amount of bound antibody correlates positively to the expression level of the hZIP1 polynucleotide in the prostate sample and control.

6. The method of claim 5, wherein said antibody is specific for a hZIP1 protein comprising the sequence of SEQ ID NO: 4.

7. The method of claim 6, wherein said antibody is specific for a hZIP1 peptide comprising the sequence of SEQ ID NO: 5.

8. The method of claim 1, wherein said prostate sample is a prostate tissue.

9. The method of claim 1, wherein said prostate sample is prostatic fluid.

10. A method of detecting prostate cancer comprising:
determining a zinc concentration in a prostate sample, wherein a decrease in said zinc concentration in the sample as compared to a zinc concentration in a control is indicative of prostate cancer.

11. The method of claim 10, said determining step comprising:
measuring zinc colorimetrically using 5-Br-PAPS [2-(5-bromo-2-pyridylazo)-5-(N-n-propyl.N-3-sulfopropylamino)phenol], 1-(2-pyridylazo)-2-naphthol or 4-(2-pyndylazo)resorcinol.

12. The method of claim 10, further comprising:
detecting a second component in the prostate sample, wherein a concentration of said second component is the same in normal and cancerous prostate glands, wherein a decrease in the ratio of zinc concentration to said second component in the prostate sample as compared to a ratio of zinc concentration to said second component in the control is indicative of prostate cancer.

13. The method of claim 12, wherein said second component is copper, calcium, iron, or cadmium.

14. The method of claim 10, said determining step comprising:
measuring zinc fluorometrically using energy dispersive x-ray fluorescence detection.

15. The method of claim 14, further comprising:
detecting a second component in the prostate sample, wherein a concentration of said second component is the same in normal and cancerous prostate glands wherein a decrease in the ratio of zinc concentration to said second component in said prostate sample as compared to the ratio of zinc concentration to said second component in the control is indicative of prostate cancer.

16. The method of claim 15, wherein said second component is copper, calcium, iron, or cadmium.

17. The method of claim 10, wherein said prostate sample is a prostate tissue.

18. The method of claim 10, wherein said prostate sample is prostatic fluid.

19. A method of detecting prostate cancer, comprising:
determining a citrate concentration in a prostate sample, wherein a decrease in said citrate concentration in the sample as compared to a citrate concentration in a control is indicative of prostate cancer.

20. The method of claim 19, said determining step comprising:
measuring citrate using a fluoroenzymatic assay.

21. The method of claim 19, said determining step comprising:
measuring citrate using an acetic anhydride/pyridine assay having a sensitivity of at least 0.5 microgram of citrate.

22. The method of claim 19, said determining step comprising:
measuring citrate using diazotized p-nitroaniline reagent and citrate lyase.

23. The method of claim 22, wherein citrate is converted to oxaloacetate.

24. The method of claim 19, further comprising:
detecting a second component in the prostate sample, wherein the concentration of said second component is the same in a normal and a cancerous prostate gland, wherein a decrease in the ratio of the citrate concentration to said second component in the prostate sample as compared to the ratio of the citrate concentration to said second component in the control is indicative of prostate cancer.

25. The method of claim 24, wherein said second component is lactate.

26. The method of claim 25, said detecting lactate comprising:
measuring lactate concentration using a fluoroenzymatic assay.

27. The method of claim 19, wherein said prostate sample is a prostate tissue.

28. The method of claim 19, wherein said prostate sample is prostatic fluid.

29. A method of detecting prostate cancer, comprising:
determining a zinc concentration and a citrate concentration in a prostate sample, wherein a decrease in said zinc and citrate concentration in the prostate sample as compared to a zinc concentration and a citrate concentration in a control is indicative of prostate cancer.

30. The method of claim 29, wherein the zinc level and the citrate level are determined using the same or different methods.

31. The method of claim 29, wherein determining the zinc level comprises measuring said zinc level fluorometrically.

32. The method of claim 31, wherein said measuring step comprises energy dispersive x-ray fluorescence detection.

33. The method of claim 29, further comprising:
detecting a second component in the prostate sample, wherein the concentration of said second component in the prostate sample is the same in a normal and a cancerous prostate gland, wherein a decrease in the ratio of the zinc concentration to said second component and a decrease in the ratio of the citrate concentration to said second component in the prostate sample as compared to the ratio of the zinc concentration to said second component and to the ratio of the citrate concentration in the control is indicative of prostate cancer.

34. The method of claim 33, wherein said second component is lactate when determining the citrate level.

35. The method of claim 33, wherein said second component is copper, calcium, iron, or cadmium when determining the zinc level.

36. The method of claim 29, wherein said prostate sample is prostate tissue.

37. The method of claim 29, wherein said prostate sample is prostatic fluid.

38. A method of treating prostate cancer, comprising:
administering an amount of a compound that increases expression of a hZIP1 gene in a prostate cell comprising said prostate cancer in an individual.

39. The method of claim 38, further comprising:
administering a second therapy selected from the group consisting of chemotherapy, radiotherapy, hormonal therapy, or cryosurgery to the individual, wherein the compound is administered before, during or after the second therapy is administered.

40. An isolated antibody directed specifically against a human ZIP1 protein or peptide fragment thereof.

41. The isolated antibody of claim 40, wherein said human ZIP1 protein comprises the sequence of SEQ ID NO: 4.

42. The isolated antibody of claim 40, wherein said human ZIP1 peptide fragment comprises the sequence of SEQ ID NO: 5.

43. An expression vector, comprising:
a genetic sequence encoding a gene or polynucleotide therefrom effective to increase uptake of zinc into a prostate cell upon expression thereof.

44. The expression vector of claim 43, wherein said genetic sequence comprises a promoter inducible by exogenous zinc.

45. The expression vector of claim 43, wherein said genetic sequence comprises SEQ ID NO: 1.

46. The expression vector of claim 43, wherein said expression vector is a plasmid or a viral vector.

47. The expression vector of claim 46, wherein said viral vector is an adenoviral vector.

48. A method of increasing zinc uptake into a prostate cell, comprising:
contacting a prostate cell with the expression vector of claim 43 in the presence of exogenous zinc.

49. The method of claim 48, wherein said prostate cell is a cancerous prostate cell in an individual, said method exhibiting a first therapeutic effect against a prostate cancer.

50. The method of claim 49, further comprising:
administering a second therapy selected from the group consisting of chemotherapy, radiotherapy, brachytherapy, hormonal therapy, or cryosurgery to the individual, wherein the expression vector is administered before, during or after administration of said second therapy.


SEQUENCE LISTING (5 sequences)

SEQ ID NO: 1 - DNA, 2445 bp, Homo sapiens
  Polynucleotide sequence of human hZIP1 gene
aggagagtcaggccaatggggccgcagttctttcttttttttttctttattcttatttttggagacagggtctcgctctgttgcccaggctggagtgcggtggtgcgatcacggttccatgcagcccccgacctcccgggctcaggtgattctcccgcctcagcaccgcgagcagctaggaccacaggcgcgagccactgcgtccggccggcgggacttatttgtcaggcggggattgggttccgccagcctaaagggaggggtaagcgccagaatatgaatcgccgggaagctgggagaaagctccgggaaaccctgagcagccaggtcgcctgctccgcccgctcccgctcccgatctctgattgctcctaactgacgtcactcccggtctgtccccgcccactcggtgctgccattggcagtcggtcgtgggtctgagagtcactggagctaccagaagcatcatggggccctggggagagccagagctcctggtgtggcgccccgaggcggtagcttcagagcctccagtgcctgtggggctggaggtgaagttgggggccctggtgctgctgctggtgctcaccctcctctgcagcctggtgcccatctgtgtgctgcgccggccaggagctaaccatgaaggctcagcttcccgccagaaagccctgagcctagtaagctgtttcgcggggggcgtctttttggccacttgtctcctggacctgctgcctgactacctggctgccatagatgaggccctggcagccttgcacgtgacgctccagttcccactgcaagagttcatcctggccatgggcttcttcctggtcctggtgatggagcagatcacactggcttacaaggagcagtcagggccgtcacctctggaggaaacaagggctctgctgggaacagtgaatggtgggccgcagcattggcatgatgggccaggggtcccacaggcgagtggagccccagcaaccccctcagccttgcgtgcctgtgtactggtgttctccctggccctccactccgtgttcgaggggctggcggtagggctgcagcgagaccgggctcgggccatggagctgtgcctggctttgctgctccacaagggcatcctggctgtcagcctgtccctgcggctgttgcagagccaccttagggcacaggtggtggctggctgtgggatcctcttctcatgcatgacacctctaggcatcgggctgggtgcagctctggcagagtcggcaggacctctgcaccagctggcccagtctgtgctagagggcatggcagctggcacctttctctatatcacctttctggaaatcctgccccaggagctggccagttctgagcaaaggatcctcaaggtcattctgctcctagcaggctttgccctgctcactggcctgctcttcatccaaatctagggggcttcaagagaggggcaggggagattgatgatcaggtgcccctgttctcccttccctcccccagttgtggggaataggaaggaaaggggaagggaaatactgaggaccaaaaagttctctgggagctaaagatagagcctttggggctatctgactaatgagagggaagtgggcagacaagaggctggccccagtcccaaggaacaagagatggtcaagtcgctagagacatatcaggggacattaggattggggaagacacttgactgctagaatcagaggttggacactatacataaggacaggctcacatgggaggctggaggtgggtacccagctgctgtggaacgggtatggacaggtcataaacctagagtcagtgtcctgttggtcctagcccatttcagcaccctgccacttggagtggacccctcctactcttcttagcgcctaccctcatacctatctccctcctcccatctcctaggggactggcgccaaatggtctctccctgccaattttggtatcttctctggcctctccagtcctgcttactcctctatttttaaagtgccaaacaaatccccttcctctttctcaaagcacagtaatgtggcactgagccctacccagcacctcagtgaagggggcctgcttgctctttattttggtcccggatcctggggtggggcagaaatattttctgggctggggtaggaggaaggttgttgcagccatctactgctgctgtaccctaggaatatggggacatggacatggtgtcccatgcccagatgataaacactgagctgccaaaacatttttttaaatacacccgaggagcccaagggggaagggcaatgcctacccccagcgttatttttggggagggagggctgtgcatagggacatattctttagaatctattttattaactgacctgttttgggacctgttacccaaataaaagatgtttctagaaaaaaaaaaaaaaaaaaa

SEQ ID NO: 2 - DNA, 20 bp, artificial sequence
  primer for human hZIP1 gene
tcagagcctccagtgcctgt

SEQ ID NO: 3 - DNA, 20 bp, artificial sequence
  primer for human hZIP1 gene
gcagcaggtccaggagacaa

SEQ ID NO: 4 - protein, 324 aa, Homo sapiens
  Sequence of human hZIP1 peptide encoded by hZIP1 gene
MGPWGEPELLVWRPEAVASEPPVPVGLEVKLGALVLLLVLTLLCSLVPICVLRRPGANHEGSASRQKALSLVSCFAGGVFLATCLLDLLPDYLAAIDEALAALHVTLQFPLQEFILAMGFFLVLVMEQITLAYKEQSGPSPLEETRALLGTVNGGPQHWHDGPGVPQASGAPATPSALRACVLVFSLALHSVFEGLAVGLQRDRARAMELCLALLLHKGILAVSLSLRLLQSHLRAQVVAGCGILFSCMTPLGIGLGAALAESAGPLHQLAQSVLEGMAAGTFLYITFLEILPQELASSEQRILKVILLLAGFALLTGLLFIQI

SEQ ID NO: 5 - protein, 14 aa, artificial sequence
  133..146: Immunogenic peptide fragment from amino acids 133-146 of human hZIP1 peptide
YKEQSGPSPLEETR